(19) United States
(12) Patent Application Publication
(10) Pub. No.: US 2004/0057944 A1
(43) Pub. Date: Mar. 25, 2004

(54) Microbial enzyme mixtures useful to treat digestive disorders

(75) Inventors: Manfred Galle, Isernhagen (DE); Peter-Colin Gregory, Hannover (DE); Andreas Potthoff, Hannover (DE); Friederike Henniges, Braunschweig (DE)

Correspondence Address: CROWELL & MORING LLP, INTELLECTUAL PROPERTY GROUP, P.O. BOX 14300, WASHINGTON, DC, 20044-4300, US

(73) Assignee: Solvay Pharmaceuticals GmbH, Hannover (DE)

(21) Appl. No.: 10/620,759

(22) Filed: Jul. 17, 2003

Related U.S. Application Data
(63) Continuation of application No. PCT/EP02/00374, filed on Jan. 16, 2002.

(30) Foreign Application Priority Data
Jan. 19, 2001 (DE) 101 02 495.9
Sep. 11, 2001 (DE) 101 44 711.6

(51) Int. Cl. A61K 38/54

(52) U.S. Cl. 424/94.2

(57) ABSTRACT

Mixtures of microbial enzymes containing a concentrated Rhizopus delemar lipase, an Aspergillus melleus protease and an Aspergillus oryzae amylase; pharmaceutical preparations containing such mixtures; and the use of such mixtures in methods of treatment and/or prophylaxis of maldigestion, especially maldigestion caused by pancreatic insufficiency, in humans or other mammals.

### CROSS REFERENCE TO RELATED APPLICATIONS

This application is a continuation of international patent application no. PCT/EP02/00374, filed Jan. 16, 2002, designating the United States of America and published in German as WO 02/060474, the entire disclosure of which is incorporated herein by reference. Priority is claimed based on Federal Republic of Germany patent application nos. DE 101 02 495.9, filed Jan. 19, 2001, and DE 101 44 711.6, filed Sep. 11, 2001.

### BACKGROUND OF THE INVENTION

The present invention relates to novel enzyme mixtures which contain a certain combination of microbial lipase, protease and amylase. Furthermore, the invention relates to pharmaceutical preparations containing these mixtures of microbial enzymes. These novel pharmaceutical preparations are particularly well suited for the treatment and/or prophylaxis of maldigestion in mammals and humans, in particular for the treatment and/or prophylaxis of maldigestion based on chronic exocrine pancreatic insufficiency.

Maldigestion in mammals and humans is usually based on a deficiency of digestive enzymes, in particular on a deficiency of endogenous lipase, but also of protease and/or amylase. The cause of such a deficiency of digestive enzymes frequently lies in a hypofunction of the pancreas (=pancreatic insufficiency), the organ which produces the most, and the most important, endogenous digestive enzymes. If the pancreatic insufficiency is pathological, this may be congenital or acquired. Acquired chronic pancreatic insufficiency may for example be ascribed to alcoholism. Congenital pancreatic insufficiency may for example be due to the congenital disease cystic fibrosis. The consequences of the deficiency of digestive enzymes may be severe symptoms of undernutrition and malnutrition, which may be accompanied by increased susceptibility to secondary illnesses.

Substitution with similarly-acting exogenous digestive enzymes or mixtures of digestive enzymes has proved effective treatment for a deficiency in endogenous digestive enzymes. Most frequently, nowadays pharmaceutical preparations which contain porcine pancreatin (=pancreatin) are used for this purpose. Such mixtures of digestive enzymes obtained from the pancreases of pigs can be used virtually ideally for enzyme substitution therapy in humans owing to the great similarity of the enzymes and accompanying substances contained therein to the contents of human pancreatic juices. Since some of the constituents of pancreatin—for example pancreatic lipase and pancreatic amylase—are sensitive to acidic pH values of less than pH 5, pancreatin preparations intended for oral administration should be coated with enteric protective layers for protection against acid-induced denaturation in the stomach. Such protective layers preserve the acid-sensitive pancreatin constituents from irreversible destruction and release their contents only after passage through the stomach in the upper region of the small intestine, where usually higher, harmless pH values—of between about pH 5.5 and pH 8—prevail. At the same time, the upper region of the small intestine, for example the duodenum, is the location at which as a rule the majority of the enzymatically broken-down food constituents is resorbed by the body.

Since pancreatin is a natural product, very considerable technical outlay is required to provide it in a uniform-quality, high-grade form. In addition, the availability of raw materials suitable for processing into pancreatin may be subject to fluctuations.

There have therefore already been attempts on various occasions to make available mixtures of digestive enzymes which are suited similarly well to pancreatin for the substitution of endogenous digestive enzymes but have improved properties compared with pancreatin.

In order to be suitable for the substitution of digestive enzymes in humans, all substitution enzymes must meet a number of requirements (cf. e.g. G. Peschke, “Active Components and Galenic Aspects of Enzyme Preparations” in: Pancreatic Enzymes in Health and Disease, editor: P. G. Lankisch, Springer Verlag Berlin, Heidelberg 1991, pages 55 to 64; hereafter cited as “Peschke”). Thus these substitution enzymes should inter alia be stable with respect to pepsin and other endogenous proteases such as pancreatic proteases. Substitution enzymes should retain their activity even in the presence of endogenous bile salts.

It is usually assumed that substitution of the endogenous lipase which is underproduced e.g. due to illness represents the most important constituent of substitution therapy for digestive enzymes in humans. However, it has been known for a relatively long time that the simultaneous substitution of underproduced protease and amylase has an additional beneficial effect on the affected patients (cf. e.g. Peschke, page 55; WO 96/38170, page 6). Pharmaceutical preparations for the treatment and/or prophylaxis of maldigestion in mammals and humans should therefore largely replace not only the lipolytic but also the proteolytic and amylolytic activities of the body. What is important here is that the different substitution enzymes contained in the pharmaceutical preparation (lipase, protease, amylase) can each develop their activity at the point of action intended therefor (this is as a rule the upper region of the small intestine) to a sufficient extent. Since under physiological conditions during or shortly after ingestion of food in the human stomach inter alia usually a higher pH value, for example pH 4-5, is present than in an empty stomach (approx. pH 1-2) and since the physiological pH value in the region of the upper intestine is usually between 5.5 and 8, digestive enzymes which have good pH stability and good pH activity in this pH range of about 4 to 8 are regarded as well suited for the substitution of digestive enzymes in humans.

Preparations are already known from European Patent Application EP 387,945 which also contain a microbial lipase in addition to a mammalian pancreas extract. Owing to the content of animal pancreatin still contained therein, such preparations cannot however be prepared by laboratory processes which are simple to standardize in always constant quality and in any quantity desired.

International Patent Application WO 96/38170, describes preparations which inter alia contain an acid-stable amylase of Aspergillus niger and optionally an acid-stable lipase of Rhizopus javanicus and which can be used as a digestion aid. However, no concrete proposals are made in this document for the substitution of the endogenous proteolytic activity. Instead, reference is merely made to the fact that there is the possibility of substituting all the other constituents of human pancreatic juice apart from lipase and amylase with porcine pancreatin. This indicates that the preparations described in WO 96/38170 are not intended or suitable for the total substitution of endogenous digestive enzymes.

Furthermore, in the dissertation by S. Scheler, title: “Multiple unit-Zubereitungen aus Aspergillus oryzae-Enzymen hoher Aktivität mit optimierter digestiver Potenz” (Multiple Unit-preparations of Aspergillus oryzae-Enzymes of Higher Activity with Optimum Digestive Potency), University of Erlangen-Nürnberg, 1995, a combination of the commercially obtainable enzymes lipase of Rhizopus oryzae, protease of Aspergillus oryzae and amylase of Aspergillus oryzae from largely pharmaceutical points of view are investigated. However, for example, the lipase used therein is not of satisfactory stability with respect to endogenous pancreatic protease.

It is clear from the above particulars that pharmaceutical preparations which are intended for total substitution of endogenous digestive enzymes of mammals and humans must contain substitution enzymes or mixtures of substitution enzymes which are carefully matched to the endogenous conditions.

### SUMMARY OF THE INVENTION

It was therefore an object of the present invention to provide improved mixtures of digestive enzymes and pharmaceutical preparations containing such mixtures for the treatment and/or prophylaxis of maldigestion in mammals and humans.

Another object of the invention was to provide mixtures of digestive enzymes which can substitute endogenous lipolytic, proteolytic and amylolytic enzyme activity.

A further object of the invention was to provide mixtures of digestive enzymes which while having high specific activity of the substitution enzymes contained therein permit use of relatively low dosage quantities.

An additional object of the invention was to provide mixtures of digestive enzymes in which the substitution enzymes (i.e., lipase, protease, and amylase), both individually and in mixtures with each other, fulfill as well as possible all the requirements made of digestive enzymes intended for human therapy in humans.

A still further object of the invention was to provide a mixture of digestive enzymes in which the enzymes have good pH stability and good pH activity in the pH range usually prevailing at the respective physiological point of action.

Yet another object of the invention is to provide a mixture of digestive enzymes in which the enzymes are readily compatible with endogenous active substances such as bile salts or endogenous proteases, for example pepsin or pancreatic proteases.

It was also an object of the invention to provide a mixture of digestive enzymes in which the constituent enzymes can be obtained in a constant quality and in any quantity desired, by production processes which are simple to standardize in relation to process and product quantity.

These and other objects have been achieved in accordance with the present invention by providing a mixture of microbial enzymes comprising:

a) a concentrated lipase of Rhizopus delemar,

b) a neutral protease of Aspergillus melleus, and

c) an amylase of Aspergillus oryzae.

Mixtures of microbial enzymes according to the invention may be contained, together with conventional auxiliaries and/or carriers, in conventional pharmaceutical preparations. These pharmaceutical preparations contain as active substances exclusively mixtures according to the invention of microbial enzymes of certain molds and are suitable for total substitution of endogenous digestive enzymes of mammals and humans. What the individual enzymes (lipase, protease, amylase) contained in the mixture of microbial enzymes according to the invention have in common is that they have good pH stability and good pH activity in the physiological to pathophysiological pH range of the digestive tract (approximately pH 4 to 8) and in particular under the conditions prevailing during or shortly after ingestion of food. The pharmaceutical preparations are furthermore distinguished by good effectiveness and good compatibility.

### BRIEF DESCRIPTION OF THE DRAWINGS

The invention will be described in further detail hereinafter with reference to the accompanying drawings in which:

FIG. 1 is graph of the pH profile of microbial “Lipase D Amano 2000”;

FIG. 2 is a graph of the pH profile of microbial protease “Prozyme 6” and

FIG. 3 is a graph of the pH profile of microbial “Amylase A1”.

### DETAILED DESCRIPTION OF THE INVENTION

The concentrated lipase of Rhizopus delemar has a specific activity of at least 1,800,000 FIP units/g (=internationally standardized enzyme activity units determined in accordance with the specifications of the “Féderation Internationale Pharmaceutique”, Belgium). The strain Rhizopus delemar is regarded as a subspecies of the strain Rhizopus oryzae. Lipases of molds of the strain Rhizopus delemar are known per se and can be obtained e.g. using known processes from culture solutions of the corresponding mold. Methods for fermenting molds and isolating the enzyme products formed by these molds are known to persons skilled in the art, for example from specialist biotechnology textbooks (cf. e.g. H. Diekmann, H. Metz, “Grundlagen und Praxis der Biotechnologie” (Fundamentals and Practice of Biotechnology), Gustav Fischer Verlag Stuttgart, New York 1991) or from specialist scientific publications. Then the isolated lipases may e.g. in known manner be freed of accompanying substances and enriched or concentrated until the specific activity desired according to the invention is achieved. Preferably the lipase (EC No. 3.1.1.3) “Lipase D Amano 2000®” (also known as “Lipase D2®”) of Rhizopus delemar from Amano Pharmaceuticals, Japan, may be used. This lipase—like natural pancreatic lipase—has a 1.3 positional specificity in relation to fatty acid glycerides. The specific activity is between about 1,800,000 FIP units/g and about 2,250,000 FIP units/g, depending on the charge. “Lipase D Amano 2000®” is distinguished by high stability in relation to pancreatic protease from pancreatin. Thus the lipolytic activity of “Lipase D Amano 2000®” in a laboratory test after two hours' action of pancreatic protease from pancreatin in a pH range of pH 6 to 8 is still at 55% of the initial activity. The pH stability of “Lipase D Amano 2000®” in a laboratory test in a pH range of pH 4 to 8 at 37° C. over a period of 120 min. was at least 70% of the initial activity.

The pH profile for a concentrated lipase of Rhizopus delemar for example is suitable as a characteristic determinant thereof. Therefore the pH profile of “Lipase D Amano 2000®” was determined as specific activity as a function of the pH value. The specific activities at the individual pH values were measured in accordance with a modification of the FIP methods to determine the activity of microbial lipases. Additionally the pH profiles were also determined in the presence of variable concentrations of bile salts.

a) Preparation of the Olive Oil Emulsion

44 g gum arabic,

115 g olive oil, and

400 ml water

were homogenized for 15 minutes in an electric mixer.

b) Preparation of the Bile Extract Solutions of Different Concentrations

| | |
|---|---|
| without bile: | 120 ml water |
| 0.5 mmol/l bile: | 120 ml water + 200 mg bile extract (FIP standard) |
| 5 mmol/l bile: | 120 ml water + 2 mg bile extract |
| 10 mmol/l bile: | 120 ml water + 4 mg bile extract |

c) Preparation of the Substrate Emulsion

| | |
|---|---|
| 480 ml | olive oil emulsion (see above) |
| 160 ml | calcium chloride solution (28.3 g CaCl₂x2 H₂O/l water) and |
| 120 ml | bile extract solution (see above) of the desired concentration |
| | were mixed. |

d) Preparation of the Enzyme Solution

50 mg “Lipase D Amano 2000®” (specific activity determined as 2,230,000 FIP units/g) was dissolved in 100 ml 1%-strength sodium chloride solution. 1 ml of this stock solution was taken and diluted to 200 ml with ultrapure water. In each case, 1 ml of the diluted stock solution (corresponding to 5.575 FIP units) was used in the following determinations.

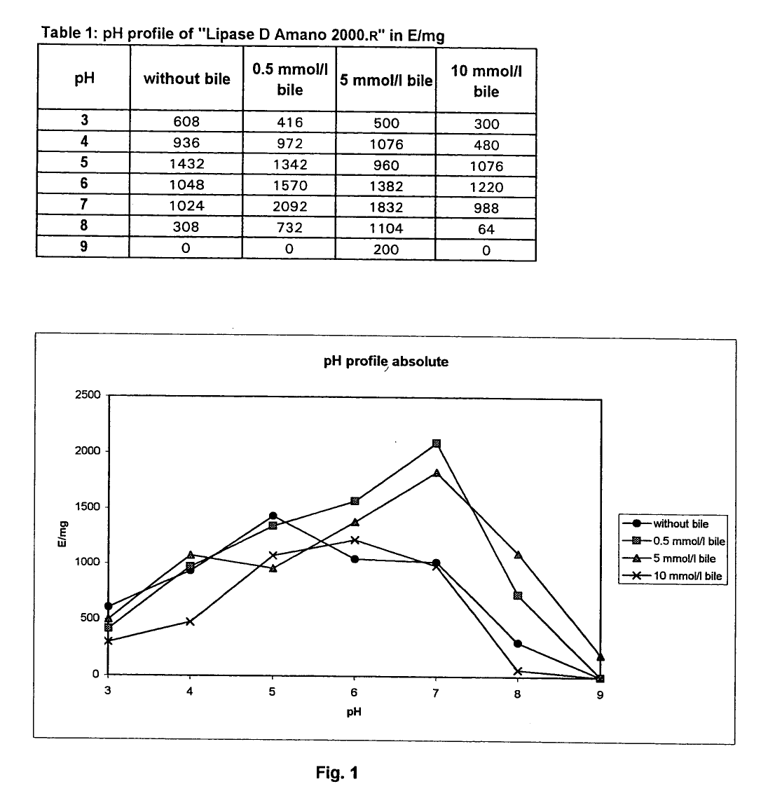

Of the above substrate emulsions, in which certain bile salt concentrations are present, samples of 19 ml were each thermostated to 37° C. pH values of 3, 4, 5, 6, 7 and 8 were then established in different samples of substrate emulsions by addition of 0.1 M NaOH or 1 M HCl. Then 1 ml of the above enzyme solution was added to each of the samples of the resulting substrate emulsions (note: in order to determine the optimum titration rate, the suitable quantity of lipase ideally contained in the enzyme solution can in principle be determined in known manner by a dilution series). Once addition had taken place, a pH stat titration with 0.1 M NaOH was performed for 10 min. Then within 30 sec. an end-point titration to pH 9 was performed in order completely to dissociate released fatty acids. The total consumption of 0.1 M NaOH required was converted into lipase activity units E: one lipase activity unit E corresponds to a consumption of 1 μmole per minute. The lipase activity units determined can be converted into units of E/mg by reference to the quantity of dry enzymes in g used each time. To draw up the pH profile, the units of E/mg for each pH value investigated and each bile salt concentration investigated are set forth in Table 1 and the values shown are plotted on a graph in FIG. 1.

The pH optimum for “Lipase D Amano 2000®” can be determined from the above pH profile as the maximum value of the lipase activity at the FIP standard bile salt concentration of 0.5 mmol/liter as about pH 7.

The neutral protease of Aspergillus melleus has a specific activity of at least 7,500 FIP units/g. Its pH optimum is between pH 6 and pH 8. Neutral proteases of molds of the strain Aspergillus melleus are known per se and can be obtained e.g. using known processes from culture solutions of the corresponding mold. Methods for fermenting molds and isolating the enzyme products formed by these molds are known to persons skilled in the art, for example from specialist biotechnology textbooks (cf. e.g. H. Diekmann, H. Metz, “Grundlagen und Praxis der Biotechnologie” (Fundamentals and Practice of Biotechnology), Gustav Fischer Verlag Stuttgart, New York 1991) or from specialist scientific publications. Then the isolated proteases may if desired in known manner be freed of accompanying substances and enriched or concentrated until the specific activity desired according to the invention is achieved.

Preferably the neutral protease “Prozyme 6®” (occasionally also referred to as “alkaline proteinase”, EC No. 3.4.21.63) of Aspergillus melleus from Amano Pharmaceuticals, Japan, may be used. This microbial protease hydrolyses 1,4-α-D-glucoside bonds of polysaccharides which contain at least three 1,4-α-D-glucose units and has a specific activity of approximately 7,800 FIP units/g. The pH stability of the protease “Prozyme 6®” in a laboratory test in a pH range of pH 5 to 8 at 37° C. over a period of 120 min. was at least 60% of the initial activity.

The pH profile for a neutral protease of Aspergillus melleus for example is suitable as a characteristic determinant thereof. Therefore the pH profile of the protease “Prozyme 6®” was determined as specific activity as a function of the pH value.

To this end, various substrate solutions were prepared, corresponding to the specifications of the FIP method for determining activity of pancreatic proteases. In a modification of the FIP specifications, a 4% hemoglobin solution is used as substrate solution instead of casein. Additionally, in a modification of the FIP specifications different pH values each of 2, 3, 4, 5, 6, 7 and 8 were established in different substrate solutions by addition of corresponding quantities of 1M NaOH or 1M HCl. Samples of “Prozyme 6®” were added to the substrate solutions.

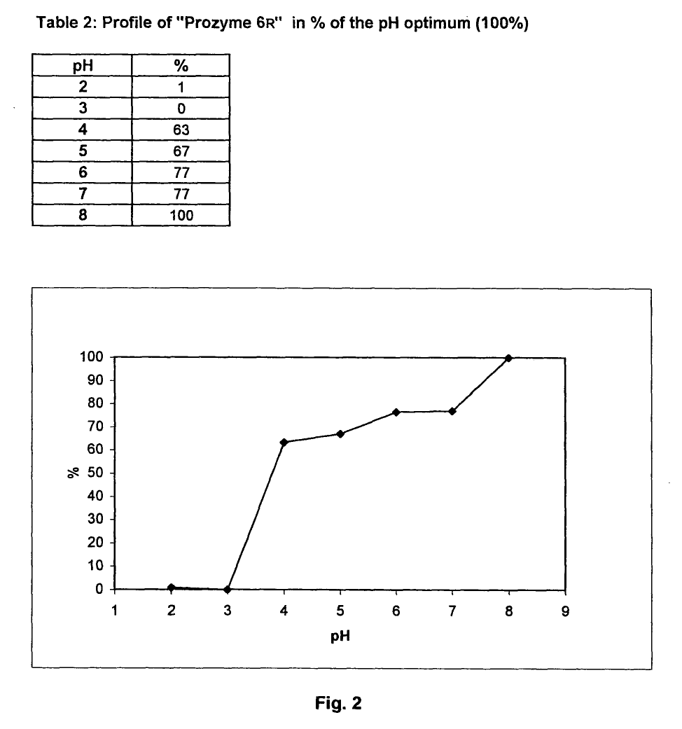

Then the protease activities of the “Prozyme 6®” samples were determined corresponding to the above specifications of the FIP in the substrate solutions of different pH values. The enzyme activities found in the individual samples were standardized to the maximum value (=100%) found in this measurement series. The measured values of the pH profile found for “Prozyme 6®” are set forth in Table 2 and are plotted on a graph in FIG. 2. “Prozyme 6®” is thus optimally effective in the physiological pH range.

The pH optimum for “Prozyme 6®” can be determined from the above pH profile as the maximum value of the protease activity as about pH 8.

The amylase used according to the invention (EC No. 3.21.1.1) of Aspergillus oryzae is an α-amylase and has a specific activity of at least 40,000 FIP units/g (measured at pH 5.8). The pH optimum lies in the pH range of pH 4 to 6.5. Amylases of molds of the strain Aspergillus oryzae are known per se and can be obtained e.g. using known processes from culture solutions of the corresponding mold. Methods for fermenting molds and isolating the enzyme products formed by these molds are known to persons skilled in the art, for example from specialist biotechnology textbooks (cf. e.g. H. Diekmann, H. Metz, “Grundlagen und Praxis der Biotechnologie” (Fundamentals and Practice of Biotechnology), Gustav Fischer Verlag, Stuttgart, New York, 1991) or from specialist scientific publications. Then the isolated amylases may if desired in known manner be freed of accompanying substances and enriched or concentrated until the specific activity desired according to the invention is achieved. Preferably the amylases “Amylase A1®” of Aspergillus melleus from Amano Pharmaceuticals, Japan and “Amylase EC®” of Aspergillus melleus from Extrakt-Chemie, Germany, may be used. “Amylase A1®” is preferred.

The microbial amylase “Amylase A1®” has a specific activity of about 52,000 FIP units/g (measured at pH 5.8). The pH stability of “Amylase A1®” in a laboratory test in a pH range of pH 5 to 8 at 37° C. over a period of 120 min. was at least 85% of the initial activity. In further laboratory tests, good stability of the “Amylase A1®” with respect to pancreatic protease from pancreatin (measured in a pH range pH 6 to 8), with respect to “Prozyme 6®” (measured in a pH range pH 4 to 8) and with respect to pepsin was noted.

The pH profile for an amylase of Aspergillus oryzae, for example, is suitable as a characteristic determinant thereof. Therefore the pH profile of “Amylase A1®” was determined as specific activity as a function of the pH value.

Various substrate solutions were prepared, corresponding to the specifications of the FIP method for determining activity of microbial amylases. In a modification of the FIP specifications in different substrate solutions by prior addition of corresponding quantities of 5 M NaOH or 5 M HCl to the acetate buffer used in accordance with the FIP method different pH values of in each case 3.25; 4; 5; 6; 6.8 and 7.4 were adjusted. Samples of “Amylase A1®” were added to the substrate solutions.

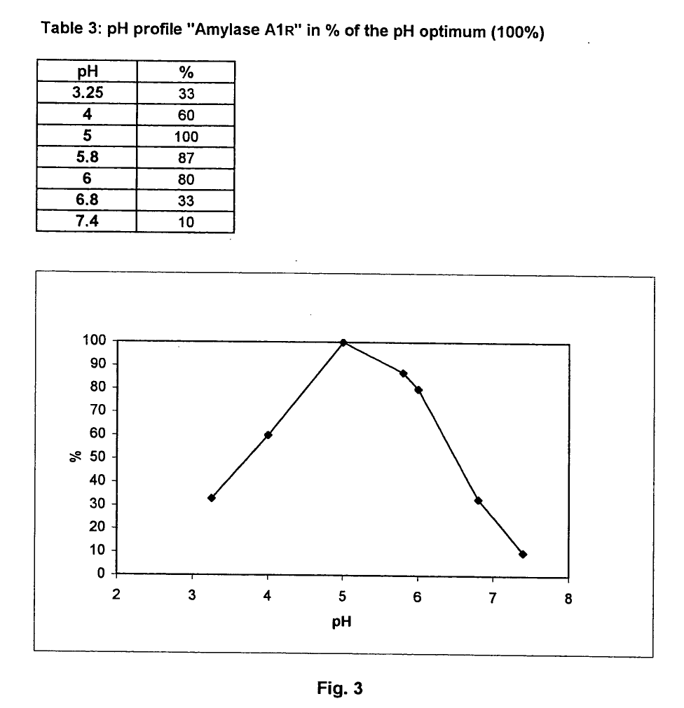

Then the amylase activities of “Amylase A1®” samples were determined corresponding to the above specifications of the FIP in substrate solutions of different pH values. The enzyme activities found in the individual samples were standardized to the maximum value (=100%) found in this measurement series. The measured values of the pH profile found for “Amylase A1®” are set forth in Table 3 and are plotted on a graph in FIG. 3.

The pH optimum for “Amylase A1®” can be determined from the above pH profile as the maximum value of the amylase activity as about pH 5.

The microbial amylase “Amylase EC®” has a specific activity of about 42,500 FIP units/g (measured at pH 5.8). In addition, small amounts of α-amylase can be detected. The pH optimum (measured in accordance with the method given above for “Amylase A1®”) is about pH 5. The pH stability of “Amylase EC®” in a laboratory test in a pH range of pH 6 to 8 at 37° C. over a period of 120 min. was at least 80% of the initial activity. In further laboratory tests, good stabilities of “Amylase EC®” with respect to pancreatic protease from pancreatin (measured in a pH range pH 6 to 8), with respect to “Prozyme 6®” (measured in a pH range pH 4 to 8) and with respect to pepsin were noted.

For the pharmaceutical preparations according to the invention, preferably solid orally administered dosage forms may be selected, for example powders, pellets or microspheres, which if desired may be filled into capsules or sachets or may be compressed to form tablets. Also liquid pharmaceutical preparations such as suspensions or solutions may be considered. The individual enzymes lipase, protease and amylase may in this case be present together or spatially separated from each other. If the individual enzymes are not spatially separated from each other, dry processing and/or storage is preferred. The pharmaceutical preparations may furthermore contain conventional auxiliaries and/or carriers. Suitable auxiliaries and/or carriers include, for example, microcrystalline celluloses, polyethylene glycols, for example PEG 4000, or alternatively lower alcohols, in particular straight-chain or branched C1-C4-alcohols such as 2-propanol, and also water.

The microbial substitution enzymes used according to the invention are distinguished by good stability over wide pH ranges and can therefore be used without further treatment (such as film-coating) directly for the preparation of orally administered pharmaceutical preparations. To this end, the individual substitution enzymes (lipase, protease and amylase) may be pelletized together or spatially separated from each other. If desired, the individual substitution enzymes may be film-coated with a suitable, known enteric layer. If not all substitution enzymes are to be enteric-coated, it is advantageous to pelletize the individual types of substitution enzymes separately from each other and to film-coat the pellets of each enzyme type separately. In particular, it may be advantageous to pelletize the protease and/or the lipase and to provide each of them with an enteric film coating individually. If desired, all three enzymes present in the enzyme mixture may also be jointly provided with an enteric film coating, or two enzymes may be provided with an enteric film coating, while one enzyme is not film-coated.

The high specific activities of the substitution enzymes used according to the invention make it possible to make available relatively small dosage forms yet with high effectiveness. For example, in one embodiment the pharmaceutical preparation may take the form of orally administered capsules of size 0. About 10,000-50,000 FIP units of lipase, 8,000 FIP units of amylase and 200 FIP units of protease may, for example, be present in such a dosage form. Advantageously, the substitution enzymes lipase, amylase and protease are present in a ratio of approx. 50-500 FIP units:40-120 FIP units:1 FIP unit.

The suitability of pharmaceutical preparations according to the invention for the treatment and/or prophylaxis of maldigestion in humans and other mammals can be demonstrated with the in vitro test model given below for determining lipid digestion:

### 1. Demonstration of Lipid Digestion in a Pig Feed Test Food

The influence of a mixture of microbial enzymes usable according to the invention on lipid catabolism in a pig feed test food also containing other food constituents was investigated. The addition of a calcium chloride solution serves to precipitate released fatty acids as calcium soaps.

A) Preparation of the Pig Feed Test Food

The constituents given below:

64.8 g “Altromin 9021®” commercial feed (from Altromin GmbH, Germany, fat content approx. 2-3%, substantially consisting of ground wheat)

3.85 g “Sojamin®” protein mixture (from Lukas Meyer, Germany)

24.5 g gum arabic (from Merck KGaA, Germany)

26.7 g soya oil (from Roth, Germany; main fat constituent; average molecular weight=932 g/mol)

were mixed with 265 ml ultrapure water and then homogenized for 15 minutes in a domestic mixer. The resulting homogenate was made up with ultrapure water to a volume of 450 ml.

B) Preparation of the Bile Extract Solution

1.35 g bile extract (FIP Standard; Lipase activation mixture) was dissolved in 50 ml ultrapure water.

C) Preparation of the Enzyme Solutions

1. Lipase Solution

63.1 mg “Lipase D Amano 2000®” from Amano Pharmaceuticals, Japan (specific activity at pH 7 determined at 1,888,137 FIP units/g) was dissolved in 10 ml ultrapure water. 250 μl of this stock solution was used for the following measurement.

2. Protease Solution

319 mg “Prozyme 6®” from Amano Pharmaceuticals, Japan (specific activity at pH 7.5 determined at 7,812 FIP units/g) was dissolved in 10 ml ultrapure water. 250 μl of this stock solution was used for the following measurement.

3. Amylase Solution

595 mg “Amylase EC®” from Extrakt-Chemie, Germany (specific activity at pH 5.8 determined at 13,466 FIP units/g) was dissolved in 10 ml ultrapure water. 1,000 μl of this stock solution was used for the following measurement.

D) Preparation of the Measurement Solution

2 ml of the above bile extract solution and in succession the above three enzyme solutions C)1. to C)3. were added to 15.5 ml of the above pig feed test food and the mixture was made up to 29 ml with ultrapure water.

E) Performance of the Measurement

The prepared measuring solution was kept at a constant temperature of 37° C. and set to pH 7 by end-point titration with 1 M NaOH. Immediately after addition of the three enzyme solutions, a pH stat titration was started for 20 min. and the consumption of 1 M NaOH was recorded every 10 sec. During the titration, 1 ml of a 4 M calcium chloride solution was metered in manually in steps of 50 μl such that a maximum reaction rate was achieved.

F) Result

The fats contained in the pig feed test food (=fatty acid triglycerides) had been hydrolysed to about 67% after 20 min. reaction time. This corresponds to more than 100% catabolism to form the physiological hydrolysis products, the 2-fatty acid monoglycerides (values above 100% are attributed to spontaneous rearrangement of the 2-fatty acid monoglycerides to form 1- and 3-fatty acid monoglycerides and subsequent lipolytic breakdown).

The good lipid digestion performance of a mixture of digestive enzymes containing the enzymes usable according to the invention can also be demonstrated in vitro on an olive-oil test food.

The particularly good suitability of the pharmaceutical preparations according to the invention for the treatment and/or prophylaxis of maldigestion in mammals and humans, in particular maldigestion based on pancreatic insufficiency, can also be demonstrated using in-vivo animal models, for example on pigs suffering from pancreatic insufficiency:

### 2. In vivo Effectiveness of an Enzyme Mixture According to the Invention on Pigs Suffering From Pancreatic Insufficiency

The tests were carried out on nine adult female Göttingen miniature pigs of the Ellegaard line (33-40 kg body weight), into each of which an ileocaecal bypass cannula had been inserted. The bypass cannula served to collect the chyme from the test animals. Six of these animals furthermore had the pancreatic duct ligated (=test animals). The other three animals retained an intact pancreatic duct and served as a control for the test results (=control animals). The test was performed with a total of three different doses of an enzyme mixture according to the invention. The following enzyme doses were administered:

Dose 1: 111,833 FIP units/meal “Lipase D Amano 2000®”

1,775 FIP units/meal “Prozyme 6®”

89,760 FIP units/meal “Amylase A1®”

Dose 2: 223,665 FIP units/meal “Lipase D Amano 2000®”

3,551 FIP units/meal “Prozyme 6®”

179,520 FIP units/meal “Amylase A1®”

Dose 3: 335,498 FIP units/meal “Lipase D Amano 2000®”

5,326 FIP units/meal “Prozyme 6®”

269,280 FIP units/meal “Amylase A1®”

Per dose, all the animals were fed, over a period of 22 days, twice daily with 250 g each time of a fat-rich test food which contained 170 g husbandry feed for miniature pigs (Altromin®, from Lukas Meyer; substantially double-ground wheat), 10 g protein concentrate (Sojamin 90®, from Lukas Meyer), 70 g soya oil (from Roth) and 0.625 g Cr₂O₃ (as non-resorbable marker, from Roth), mixed with 1 liter of water. Additionally the individual enzymes of the enzyme mixture according to the invention were admixed in the corresponding quantity to the feed of only the test animals shortly before feeding. Additionally, a series of tests was carried out with five of the test animals, in which no enzyme mixture was added to their test feed. The results obtained in this series of tests are given below as “zero values”. In each case on the 20th to 22nd days of the investigation period, chyme samples were taken from the bypass cannula of the test animals over a period of 12 hours, and these were investigated in terms of their content of crude fat, crude protein and starch. The feeding tests and their evaluation were carried out in known manner (cf. P. C. Gregory, R. Tabeling, J. Kamphues, “Biology of the Pancreas in Growing Animals”; Developments in Animal and Veterinary Sciences 28 (1999) 381-394, Elsevier, Amsterdam; editors: S. G. Pierzynowski and R. Zabielski).

The apparent precaecal digestibility of crude fat, crude protein and starch in the test animals determined in the above in-vivo test is given in Table A below in each case in percent, relative to the absolute quantity of fat, protein and starch originally fed. The values given as “precaecal digestibility” correspond to the “apparent precaecal digestibility”, which differ from the actual precaecal digestibility in that they may also contain small amounts of endogenous contents of the substances investigated, for example endogenous proteins. The precaecal digestibility values were determined using the formula given below from the chyme of the test animals in accordance with the marker method:

precaecal digestibility sV

$$sV ( \% ) = 100 - ( \frac{\% indicator in the feed}{\% indicator in the chyme} \times \frac{\% nutrient in the chyme}{\% nutrient in the feed} \times 100 )$$

Table A:

Determination of the precaecal digestibility of crude fat, crude protein and starch in the test animals in vivo

Precaecal digestibility (%)

| | Crude fat | Crude protein | Starch |
|---|---|---|---|
| Zero values | 29.0 +/− 9.8 | 33.7 +/− 5.2 | 63.8 +/− 6.7 |
| Test animals - dose 1 | 43.5 +/− 9.9 | 56.3 +/− 4.5 | 71.9 +/− 9.3 |
| Test animals - dose 2 | 52.1 +/− 8.3 | 64.0 +/− 3.7 | 74.2 +/− 5.8 |
| Test animals - dose 3 | 55.3 +/− 8.0 | 68.7 +/− 3.3 | 81.6 +/− 3.7 |
| Control animals | 97.6 +/− 0.02 | 82.3 +/− 1.5 | 96.9 +/− 0.5 |

All values are given as mean values with standard deviations.

It is clear from the test results given that by administering an enzyme mixture according to the invention a significant improvement in the digestibility of fats, proteins and carbohydrates is achieved in pigs suffering from pancreatic insufficiency and that this improvement is dependent on dose.

### EXAMPLE I

Pellets having a diameter of 0.7-1.4 mm were produced in known manner from 400 g “Lipase D Amano 2000®”, 400 g PEG 4000 and 1,200 g “Vivapur®” (=microcrystalline cellulose) with the addition of a little 2-propanol and water.

Pellets having a diameter of 0.7-1.7 mm were produced in known manner from 7,000 g “Amylase A1®”, 2,000 g PEG 4000 and 1,000 g “Vivapur®” with the addition of a little 2-propanol and water.

Pellets having a diameter of 0.7-1.7 mm were produced in known manner from 1,750 g “Prozyme 6®”, 500 g PEG 4000 and 250 g “Vivapur®” with the addition of a little 2-propanol and water.

From the pellets produced above, 32 mg lipase pellets, 325 mg amylase pellets and 40 mg protease pellets, respectively, were filled into a size 0 gelatine capsule. A dosage form with the following activities per capsule was obtained:

| | | |
|---|---|---|
| Lipase | approx. | 10,000 FIP units |
| Protease | approx. | 200 FIP units |
| Amylase | approx. | 8,000 FIP units |

The foregoing description and examples have been set forth merely to illustrate the invention and are not intended to be limiting. Since modifications of the described embodiments incorporating the spirit and substance of the invention may occur to persons skilled in the art, the invention should be construed broadly to include all variations within the scope of the appende claims and equivalents thereof.

What is claimed is:

1. An enzyme mixture comprising:
a) a concentrated lipase of Rhizopus delemar,
b) a neutral protease of Aspergillus melleus, and
c) an amylase of Aspergillus oryzae.

2. An enzyme mixture according to claim 1, wherein the lipase has a specific activity of at least 1,800,000 FIP units/gram.

3. An enzyme mixture according to claim 1, wherein the protease has a specific activity of at least 7,500 FIP units/gram.

4. An enzyme mixture according to claim 1, wherein the protease has a pH optimum between pH 6 and pH 8.

5. A pharmaceutical preparation comprising an effective digestive activity improving amount of a an enzyme mixture according to claim 1, and at least one carrier or adjuvant.

6. A preparation according to claim 5, wherein the preparation is in a form selected from the group consisting of powder, pellets, microspheres, capsules, sachets, tablets, liquid suspensions and liquid solutions.

7. A preparation according to claim 5, wherein at least one enzyme selected from the group consisting of lipase, protease and amylase, is in individually pelletized form.

8. A preparation according to claim 5, wherein at least one enzyme selected from the group consisting of lipase, protease and amylase, is film-coated with an enteric layer.

9. A preparation according to claim 8, wherein the protease is in individually pelletized form and film-coated with an enteric layer.

10. A preparation according to claim 8, wherein the lipase is in individually pelletized form and film-coated with an enteric layer.

11. A preparation according to claim 8, wherein the protease and the lipase are in individually pelletized form and film-coated with an enteric layer.

12. A preparation according to claim 5, wherein the enzymes are present in a lipase:amylase:protease ratio of 50-500 FIP units lipase:40-120 FIP units amylase:1 FIP unit protease.

13. A preparation according to claim 5, which contains per dosage unit at least 10,000 FIP units lipase, 8,000 FIP units amylase, and 200 FIP units protease.

14. A method of inhibiting maldigestion in a mammal comprising administering to said mammal an effective digestion improving amount of an enzyme mixture according to claim 1.

15. A method according to claim 14, wherein said mammal is a human.

16. A method according to claim 14, wherein the maldigestion is caused by pancreatic insufficiency.

17. A method of inhibiting maldigestion in a mammal comprising administering to said mammal an effective digestion improving amount of a concentrated lipase of Rhizopus delemar, which has a specific activity of at least 1,800,000 FIP units/gram.

18. A method according to claim 17, wherein said mammal is a human.
